(19) United States
(12) Patent Application Publication
(10) Pub. No.: US 2025/0177459 A1
(43) Pub. Date: Jun. 5, 2025

(54) NUTRITIONAL SUPPLEMENTS AND METHODS OF NUTRITIONAL SUPPLEMENTATION AFFECTING WEIGHT LOSS

(71) Applicant: AMARE GLOBAL, Lehi, UT (US)

(72) Inventor: Shawn M. TALBOTT, Plymouth, MA (US)

(21) Appl. No.: 18/729,156

(22) PCT Filed: Jan. 13, 2023
(86) PCT No.: PCT/US2023/010833

§ 371 Date: Jul. 15, 2024

Related U.S. Application Data
(63) Continuation-in-part of application No. 18/097,111, filed on Jan. 13, 2023.
(60) Provisional application No. 63/299,353, filed on Jan. 13, 2022.

(51) Int. Cl. A61K 35/747; A61K 31/715; A61K 35/00; A61K 35/745; A61K 36/752; A61K 36/899; A61P 3/04

(52) U.S. Cl. CPC A61K 35/747; A61K 31/715; A61K 35/745; A61K 36/752; A61K 36/899; A61P 3/04; A61K 2035/115

(57) ABSTRACT

Nutritional supplements and methods of nutritional supplementation are described for increasing weight loss in a subject. In some embodiments, the nutritional supplement includes a probiotic such as Lactobacillus, a postbiotic such as heat-treated Bifidobacterium, a prebiotic such as a fiber, and a phytobiotic such as a plant extract.

### CROSS-REFERENCE TO RELATED APPLICATIONS

This International patent application claims priority to U.S. patent application Ser. No. 18/097,111, filed Jan. 13, 2023, which claims priority to U.S. Provisional Patent Application No. 63/299,353, filed Jan. 13, 2022, the disclosures of which are hereby incorporated by reference in their entireties.

### FIELD OF INVENTION

The following relates generally to nutritional supplements and to methods of nutritional supplementation of subjects. More specifically, the following relates to nutritional supplements affecting weight loss and to methods of supplementation of subjects in need thereof.

### BACKGROUND

In recent years, research has compiled evidence that the gut microbiome of a subject may influence the communication between that subject's central and enteric nervous systems. This communication appears to travel both directions, and an imbalance of a subject's gut flora has been associated with a variety of complaints, ranging from minor discomforts to significant central nervous system and gastrointestinal disorders. Specifically, a healthy gut microbiome may improve mental health and weight loss.

Improved nutritional supplements and methods of nutritional supplementation to improve weight loss in patients remain desirable.

### SUMMARY

This disclosure describes improved nutritional supplements and methods of nutritional supplementation that affect the gut-brain axis and/or affect weight loss. Generally, the described techniques provide for unique nutritional supplements and methods of their use to increase weight loss in a subject and to positively influence that subject's health, metabolism, and mood.

In some aspects, a nutritional supplement is described.

In some embodiments, the nutritional supplement includes a probiotic comprising at least one species of probiotic bacterium, a prebiotic comprising fiber configured to grow the probiotic, a phytonutrient configured to regulate stress in a subject through the dopamine and adenosine pathways, and a postbiotic configured to increase weight loss.

In some embodiments, the nutritional supplement includes a probiotic comprising at least one species of probiotic bacterium, a prebiotic comprising fiber configured to grow the probiotic, and a phytonutrient configured to regulate stress in a subject through the dopamine and adenosine pathways.

In some aspects, a method of increasing weight loss in a subject is described. The method may include administering to the subject an effective amount of a nutritional supplement including a probiotic comprising at least one species of probiotic bacterium, a prebiotic comprising fiber configured to grow the probiotic, and a phytonutrient configured to regulate stress in a subject through the dopamine and adenosine pathways.

The scope of this disclosure shall not be limited by this summary section or the following detailed description and examples; the scope of the invention shall be determined solely by the claims as issued.

### DETAILED DESCRIPTION

This application describes compositions useful as improved nutritional supplements and methods of their use in supplementing the nutrition of patients to improve weight loss and to provide positive changes in health, metabolism, and mood.

Amounts, concentrations, and other numerical data may be presented herein in a range format. Such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, an amount of from 1 mg to 200 mg should be interpreted to include not only the explicitly recited limits of 1 mg and about 200 mg, but also to include individual amounts such as 2 mg, 3 mg, 4 mg, and sub-ranges such as 10 mg to 50 mg, 20 mg to 100 mg, etc. Unless otherwise stated, all ranges include both endpoints.

The supplement compositions of the present disclosure may be administered in a variety of suitable dosage forms, including, without limitation, tablets, capsules, granules, powders, liquids, liposome inclusions, ointments, gels, external powders, sprays, inhalable powders, injectable preparations (solutions, suspensions, emulsions, solids to be dissolved when used, etc.), eye drops, eye ointments, suppositories, and the like can be selected appropriately depending on the administration method, and the compositions of the present disclosure can be accordingly formulated. Formulation in general is described in references including Comprehensive Medicinal Chemistry, Volume 5, Editor Hansch et al, Pergamon Press 1990.

As used herein, “effective amount” refers to an amount of a substance which is sufficient to achieve its intended purpose or effect. Various biological factors may affect the ability of a delivered substance to perform its intended task. Therefore, an “effective amount” may be dependent on such biological factors. An effective amount of a compound for treating a disorder is an amount that is sufficient to ameliorate, or in some manner reduce a symptom or stop or reverse progression of a condition. Such amount may be administered as a single dosage or may be administered according to a regimen whereby it is effective. The achievement of therapeutic effects may be measured by a physician or other qualified medical personnel using evaluations known in the art, (for example with testosterone supplementation therapy, physical examination, blood and saliva tests may be used), it is recognized that individual variation and response to treatments may make the achievement of therapeutic effects a subjective decision.

As used herein, “administration,” and “administering” may be used interchangeably, and refer to the act of presenting, applying, or introducing a composition to a subject to achieve a desired physiological or psychological response.

As used herein, amelioration of the symptoms of a particular disorder by administration of a particular composition refers to any lessening, whether permanent or temporary, lasting or transient that can be attributed to or associated with administration of the composition.

In some aspects, a nutritional supplement for improving weight loss, health, metabolism, and mood in a subject is described. The nutritional supplement may include a plurality of prebiotics, probiotics, postbiotics, and phytobiotics that improve weight loss, health, metabolism, and mood in the subject. In the illustrated embodiment, the nutritional supplement includes at least three primary ingredients, and, in some embodiments, the nutritional supplement includes at least four primary ingredients. The primary ingredients are combined in specific ratios to synchronize with all parts of the gut-brain axis through prebiotics, probiotics, postbiotics, and phytobiotics to improve mental wellness and physical composition. Accordingly, the nutritional supplements described herein are natural weight loss products that operate through a synergistic 4-part process that automates, utilizes, and synchronizes all parts of the gut-brain axis through prebiotics, probiotics, postbiotics, and phytobiotics to improve mental wellness and physical composition.

In the exemplary embodiment, the nutritional supplement includes a first primary ingredient including a probiotic that lowers symptoms of stress and anxiety, as well as biomarker cortisol. Specifically, in the exemplary embodiment, the probiotic includes Lactobacillus plantarum DR7. Lactobacillus plantarum DR7 works via the gut-brain axis to optimize the serotonin pathway and gut modulation where Lactobacillus plantarum DR7 influences the norepinephrine pathway with increased dopamine levels. Lactobacillus plantarum DR7 may modulate tryptophan-serotonin which could be one of several mechanisms involved demonstrating that Lactobacillus plantarum DR7 lowers symptoms of stress and anxiety, as well as biomarker cortisol. In alternative embodiments, the nutritional supplement may include any probiotic that enables the nutritional supplement to operate as described herein.

In the exemplary embodiment, the nutritional supplement includes a second primary ingredient including a prebiotic that delivers fiber that fosters the flourishing and growth of probiotics in the microbiome and beneficial bacteria in the intestines. Specifically, in the exemplary embodiment, the prebiotic includes acacia gum. Acacia gum is an all-natural, organic, and GMO-free prebiotic ingredient sourced from acacia trees in Africa. Acacia gum is sustainable and delivers fiber enrichment to the patient that helps foster the flourishing of probiotics in the microbiome. Acacia gum has a prebiotic effect that has synergistic effects with specific bacterial strains such as Bifidobacterium lactis: 1 (“BPL1”). Acacia gum helps to restore the gut barrier and improve gut diversity through the mechanism of providing probiotics fuel. Furthermore, acacia gum increases satiety and lowers peak blood glucose response in healthy subjects. In some embodiments, the nutritional supplement may include any prebiotic that enables the nutritional supplement to operate as described herein.

In the exemplary embodiment, the nutritional supplement includes a third primary ingredient including a phytonutrient that regulates stress through the dopamine and adenosine pathways. Specifically, in the exemplary embodiment, the phytonutrient includes orange peel (Citrus sinensis (L.)) or an extract thereof. Orange peel is species of beneficial compound derived from plant sources called phytobiotics. Orange peel regulates stress through the dopamine and adenosine pathways. Orange peel is shown to reduce the feelings of stress by 50%, 53% of global stress, and to inhibit the stress response receptors in subjects. Stress is a major factor in cortisol and obesity and modulating these pathways is a key contributor to overall mental wellness and weight management. In some embodiments, the nutritional supplement may include any phytonutrient that enables the nutritional supplement to operate as described herein.

In the exemplary embodiment, the nutritional supplements described herein include three primary ingredients. In some embodiments, the nutritional supplement may include four primary ingredients. For example, in some embodiments, the nutritional supplement may further include a postbiotic. Specifically, in the exemplary embodiment, the postbiotic includes heat-treated Bifidobacterium lactis: 1 (“HT BPL1”). HT BPL1 is a vegan, heat-treated postbiotic that aids in weight management and support metabolic health. HT BPL1 reduces visceral fat and cholesterol levels and has anti-inflammatory properties.

Certain heat-treated postbiotics also carry nutrients that confer health benefits for weight loss and management. For example, after 3 months of use, subjects who received HT BPL1 experienced significant reductions in waist circumference and abdominal visceral fat compared to placebo. Waist reductions of 1.75 cm and 1.9 cm were seen when subjects were administered heat killed HT BPL1. A synergistic effect exists when combining functional fibers like acacia gum with HT BPL1 where a 35% reduction in visceral fat, as compared to declines of 18.7% and 12.7% which is 3.5 lbs per 10 lbs of visceral fat.

In some embodiments, the nutritional supplement includes up to 500 milligrams (“mg”) of the probiotic, the prebiotic, and the phytonutrient. Specifically, the nutritional supplement includes 20-70 mg of the probiotic, 80-120 mg of the prebiotic, and 100-150 mg of the phytonutrient. More specifically, in the exemplary embodiment, the nutritional supplement includes about 50 mg of the probiotic, about 100 mg of the prebiotic, and about 125 mg of the phytonutrient. In some embodiments, the nutritional supplement may include any amount of the probiotic, the prebiotic, and the phytonutrient that enables the nutritional supplement to operate as described herein.

In some embodiments, the nutritional supplement includes 0-500 milligrams (mg) of the probiotic, the prebiotic, the phytonutrient, and the postbiotic. Specifically, the nutritional supplement includes 20-70 mg of the probiotic, 80-120 mg of the prebiotic, 100-150 mg of the phytonutrient, and 10-50 mg of the postbiotic. More specifically, in the exemplary embodiment, the nutritional supplement includes about 50 mg of the probiotic, about 100 mg of the prebiotic, about 125 mg of the phytonutrient, and about 34 mg of the postbiotic. In some embodiments, the nutritional supplement may include any amount of the probiotic, the prebiotic, the phytonutrient, and the postbiotic that enables the nutritional supplement to operate as described herein.

In some aspects, a method of increasing weight loss in a subject is described. The method may include administering to the subject an effective amount of a nutritional supplement including a probiotic comprising at least one species of probiotic bacterium, a prebiotic comprising fiber configured to grow the probiotic, and a phytonutrient configured to regulate stress in a subject through the dopamine and adenosine pathways. The nutritional supplement may further include a postbiotic configured to increase weight loss.

Various aspects on the disclosure relate to a nutritional supplement, comprising one, two, three, or each of a probiotic, a prebiotic, a phytobiotic, and a postbiotic.

In some embodiments, the probiotic is a species of Lactobacillus. In some specific embodiments, the probiotic is Lactobacillus plantarum. In some very specific embodiments, the probiotic is Lactobacillus plantarum DR7.

In some embodiments, the prebiotic is fiber. In some specific embodiments, the prebiotic is acacia gum.

In some embodiments, the phytobiotic is a plant extract. In some specific embodiments, the phytobiotic is orange peel extract.

In this disclosure, the terms phytonutrient and phytobiotic are used interchangeably.

In some embodiments, the postbiotic is a species of Bifidobacterium. In some specific embodiments, the postbiotic is Bifidobacterium lactis. In some even more specific embodiments, the postbiotic is Bifidobacterium lactis 1 (“BPL1”). In some very specific embodiments, the postbiotic is heat-treated Bifidobacterium lactis 1 (“HT BPL1”).

In some embodiments, the nutritional supplement comprises 20 to 70 milligrams of the probiotic. In some specific embodiments, the nutritional supplement comprises 20 to 70 milligrams of Lactobacillus plantarum DR7.

In some embodiments, the nutritional supplement comprises 80 and 120 milligrams of the prebiotic. In some specific embodiments, the nutritional supplement comprises 80 and 120 milligrams of acacia gum.

In some embodiments, the nutritional supplement comprises 100 and 150 milligrams of the phytobiotic. In some specific embodiments, the nutritional supplement comprises 100 and 150 milligrams of orange peel extract.

In some embodiments, the nutritional supplement comprises 10 and 50 milligrams of the postbiotic. In some specific embodiments, the nutritional supplement comprises 10 and 50 milligrams of heat-treated Bifidobacterium lactis 1 (“HT BPL1”).

In some embodiments, the nutritional supplement comprises bamboo leaf extract.

Various aspects of this disclosure relate to a method to promote weight loss in a human subject.

In some embodiments, the method comprises administering a nutritional supplement as described anywhere in this disclosure to the human subject. In some specific embodiments, the method comprises administering an effective amount of a nutritional supplement as described anywhere in this disclosure to the human subject.

In some embodiments, the administering is oral administering.

In some embodiments, the administering is self-administering that is performed by the human subject.

In some embodiments, the effective amount of the nutritional supplement is effective to cause weight loss in the human subject.

In some embodiments, the nutritional supplement comprises a probiotic bacterium; a postbiotic bacterium; a phytobiotic; and a prebiotic comprising fiber that the probiotic and/or postbiotic bacterium can metabolize as a carbon source.

In some embodiments, the probiotic is a species of Lactobacillus. In some specific embodiments, the probiotic is Lactobacillus plantarum. In some very specific embodiments, the probiotic is Lactobacillus plantarum DR7.

In some embodiments, the prebiotic is fiber. In some specific embodiments, the prebiotic is acacia gum.

In some embodiments, the phytobiotic is a plant extract. In some specific embodiments, the phytobiotic is orange peel extract.

In some embodiments, the postbiotic is a species of Bifidobacterium. In some specific embodiments, the postbiotic is Bifidobacterium lactis. In some even more specific embodiments, the postbiotic is Bifidobacterium lactis 1 (“BPL1”). In some very specific embodiments, the postbiotic is heat-treated Bifidobacterium lactis 1 (“HT BPL1”).

In some embodiments, the nutritional supplement comprises the probiotic bacterium in an amount that is effective to inhibit inflammation in the human subject.

In some embodiments, the effective amount is effective to inhibit inflammation in the human subject.

In some embodiments, the nutritional supplement comprises 20 to 70 milligrams of the probiotic. In some specific embodiments, the nutritional supplement comprises 20 to 70 milligrams of Lactobacillus plantarum DR7.

In some embodiments, the nutritional supplement comprises the prebiotic in an amount that is effective to inhibit appetite in the human subject.

In some embodiments, the effective amount is effective to inhibit appetite in the human subject.

In some embodiments, the nutritional supplement comprises 80 and 120 milligrams of the prebiotic. In some specific embodiments, the nutritional supplement comprises 80 and 120 milligrams of acacia gum.

In some embodiments, the nutritional supplement comprises the phytobiotic in an amount that is effective to inhibit stress in the human subject.

In some embodiments, the effective amount is effective to inhibit stress in the human subject.

In some embodiments, the nutritional supplement comprises the phytobiotic in an amount that is effective to improve sleep quality in the human subject.

In some embodiments, the effective amount is effective to improve sleep quality in the human subject.

In some embodiments, the nutritional supplement comprises the phytobiotic in an amount that is effective to modulate dopamine in the human subject.

In some embodiments, the effective amount is effective to modulate dopamine in the human subject.

In some embodiments, the nutritional supplement comprises the phytobiotic in an amount that is effective to modulate adenosine in the human subject.

In some embodiments, the effective amount is effective to modulate adenosine in the human subject.

In some embodiments, the nutritional supplement comprises 100 and 150 milligrams of the phytobiotic. In some specific embodiments, the nutritional supplement comprises 100 and 150 milligrams of orange peel extract.

In some embodiments, the nutritional supplement comprises the postbiotic in an amount that is effective to cause weight loss in the human subject.

In some embodiments, the nutritional supplement comprises 10 and 50 milligrams of the postbiotic. In some specific embodiments, the nutritional supplement comprises 10 and 50 milligrams of heat-treated Bifidobacterium lactis 1 (“HT BPL1”).

In some embodiments, the postbiotic is heat-treated Bifidobacterium lactis 1 (“HT BPL1”).

In some embodiments, the nutritional supplement comprises one or more of plant cellulose, microcrystalline cellulose, maltodextrin, vegetable juice, and bamboo leaf extract.

In some embodiments, the nutritional supplement comprises about 50 milligrams of Lactobacillus plantarum DR7; about 100 milligrams of acacia gum; about 125 milligrams of orange peel extract; and about 34 milligrams of Bifidobacterium lactis 1 (“BPL1”).

In some embodiments, the human subject has an initial weight prior to the administering; the human subject has a final weight following the administering; and the final weight no more than 98 percent of the initial weight. In some specific embodiments, the final weight no more than 97 percent of the initial weight. In some very specific embodiments, the final weight no more than 96 percent of the initial weight.

In some embodiments, the human subject has an initial weight prior to the administering; the human subject has a final weight following the administering; and the final weight is at least 2 kilograms less than the initial weight. In some specific embodiments, the final weight is at least 3 kilograms less than the initial weight. In some very specific embodiments, the final weight is at least 4 kilograms less than the initial weight.

In some embodiments, the method comprises administering the nutritional supplement to the human subject once or twice daily for at least 4 weeks.

Various aspects of this disclosure relate to a nutritional supplement, comprising: 20 to 70 milligrams of Lactobacillus plantarum DR7;80 and 120 milligrams of acacia gum; 100 and 150 milligrams of orange peel extract; and 10 and 50 milligrams of heat-treated Bifidobacterium lactis 1 (“HT BPL1”).

In some embodiments, the nutritional supplement further comprises bamboo leaf extract.

Various aspects of this disclosure relate to a nutritional supplement for use to promote weight loss in a human subject, comprising a probiotic bacterium; a postbiotic bacterium; a phytobiotic; and a prebiotic comprising fiber that the probiotic and/or postbiotic bacterium can metabolize as a carbon source.

In some embodiments, the probiotic is a species of Lactobacillus. In some specific embodiments, the probiotic is Lactobacillus plantarum. In some very specific embodiments, the probiotic is Lactobacillus plantarum DR7.

In some embodiments, the prebiotic is fiber. In some specific embodiments, the prebiotic is acacia gum.

In some embodiments, the phytobiotic is a plant extract. In some specific embodiments, the phytobiotic is orange peel extract.

In some embodiments, the postbiotic is a species of Bifidobacterium. In some specific embodiments, the postbiotic is Bifidobacterium lactis. In some even more specific embodiments, the postbiotic is Bifidobacterium lactis 1 (“BPL1”). In some very specific embodiments, the postbiotic is heat-treated Bifidobacterium lactis 1 (“HT BPL1”).

In some embodiments, the nutritional supplement comprises 20 to 70 milligrams of the probiotic. In some specific embodiments, the nutritional supplement comprises 20 to 70 milligrams of Lactobacillus plantarum DR7.

In some embodiments, the nutritional supplement comprises 80 and 120 milligrams of the prebiotic. In some specific embodiments, the nutritional supplement comprises 80 and 120 milligrams of acacia gum.

In some embodiments, the nutritional supplement comprises 100 and 150 milligrams of the phytobiotic. In some specific embodiments, the nutritional supplement comprises 100 and 150 milligrams of orange peel extract.

In some embodiments, the nutritional supplement comprises 10 and 50 milligrams of the postbiotic. In some specific embodiments, the nutritional supplement comprises 10 and 50 milligrams of heat-treated Bifidobacterium lactis 1 (“HT BPL1”).

In some embodiments, the probiotic is Lactobacillus plantarum DR7.

In some embodiments, the postbiotic is heat-treated Bifidobacterium lactis 1 (“HT BPL1”).

In some embodiments, the nutritional supplement comprises plant cellulose.

In some embodiments, the nutritional supplement comprises microcrystalline cellulose.

In some embodiments, the nutritional supplement comprises maltodextrin.

In some embodiments, the nutritional supplement comprises vegetable juice.

In some embodiments, the nutritional supplement comprises bamboo leaf extract.

In some embodiments, the nutritional supplement comprises about 50 milligrams of Lactobacillus plantarum DR7.

In some embodiments, the nutritional supplement comprises about 100 milligrams of acacia gum.

In some embodiments, the nutritional supplement comprises about 125 milligrams of orange peel extract.

In some embodiments, the nutritional supplement comprises about 34 milligrams of Bifidobacterium lactis 1 (“BPL1”).

In some embodiments, the nutritional supplement is for use to inhibit appetite in the human subject.

In some embodiments, the nutritional supplement is for use to inhibit inflammation in the human subject.

In some embodiments, the nutritional supplement is for use to promote at least 4 kilograms of weight loss in a human subject upon oral administration at a rate of once or twice a day over a time period of 4 weeks.

In some embodiments, the nutritional supplement is for use to improve mood in the human subject.

### EXAMPLES

### Experimental Design

A study was conducted to explore the effects of supplementation using the nutritional supplements and methods of supplementation according to the present disclosure. A test group of 73 subjects, including 20 men and 53 women, were recruited to participate in the study. The subjects weighed on average 173 pounds with 32% body fat.

The trial subjects underwent nutritional supplementation with a nutritional supplement. The nutritional supplement included a blend of Lactobacillus plantarum DR7, acacia gum, orange peel, and Bifidobacterium lactis: 1.

Several physiological parameters were measured at the beginning of the study, including: weight, body fat percentage, waist circumference, and visceral fat index. Each subject's gut bacteria were characterized. Baseline measurements of subject mood state or psychological mood state were also taken of each subject.

Subjects then received the supplement once-daily for a period of four weeks, following which the weight, body fat percentage, waist circumference, and visceral fat index were measured again. As before the study, each subject's gut bacteria were characterized. Lastly, updated measurements of subject mood state were also taken.

The results indicate that 97% of subjects lost weight. The average weight loss was 4.0% of initial body weight. The highest weight loss was a total of 12 lbs. lost in 4 weeks. The average fat loss was 5.2% of initial body fat. The highest amount of body fat lost was a total of 10 lbs. lost in 4 weeks. Additionally, 100% of subjects reported overall improvements in the measurements of mood state taken at the end of the four-week period of supplementation. Notably, all of these reported mood state measurements moved in a positive direction, with reductions in depression, confusion, fatigue, anger, and tension, and increases in vigor and global mood state/well-being.

As used herein, including in the claims, “or” as used in a list of items (e.g., a list of items prefaced by a phrase such as “at least one of” or “one or more of”) indicates an inclusive list such that, for example, a list of at least one of A, B, or C means A or B or C or AB or AC or BC or ABC (i.e., A and B and C).

The description set forth herein, in connection with the example, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

1. A nutritional supplement, comprising:
20 to 70 milligrams of Lactobacillus plantarum;
80 and 120 milligrams of acacia gum;
100 and 150 milligrams of orange peel extract; and
10 and 50 milligrams of Bifidobacterium lactis.

2. The nutritional supplement of claim 1, further comprising bamboo leaf extract.

3. A nutritional supplement for promoting weight loss in a human subject, the nutritional supplement comprising:
a probiotic bacterium;
a postbiotic bacterium;
a phytobiotic; and
a prebiotic comprising fiber that the probiotic and/or postbiotic bacterium can metabolize as a carbon source.

4. The nutritional supplement of claim 3, wherein:
the probiotic bacterium is Lactobacillus plantarum;
the prebiotic is acacia gum;
the phytobiotic is orange peel extract; and
the postbiotic bacterium is Bifidobacterium lactis.

5. The nutritional supplement of claim 3, comprising 20 to 70 milligrams of the probiotic bacterium.

6. The nutritional supplement of claim 3, comprising 80 and 120 milligrams of the prebiotic.

7. The nutritional supplement of claim 3, comprising 100 and 150 milligrams of the phytobiotic.

8. The nutritional supplement of claim 3, comprising 10 and 50 milligrams of the postbiotic.

9. The nutritional supplement of claim 1, wherein the probiotic bacterium is Lactobacillus plantarum DR7.

10. The nutritional supplement of claim 1, wherein the postbiotic is heat-treated Bifidobacterium lactis 1 (“HT BPL1”).

11. The nutritional supplement of claim 1, further comprising one or more of plant cellulose, microcrystalline cellulose, maltodextrin, vegetable juice, and bamboo leaf extract.

12. The nutritional supplement of claim 1, comprising about 50 milligrams of Lactobacillus plantarum DR7;
about 100 milligrams of acacia gum;
about 125 milligrams of orange peel extract; and
about 34 milligrams of Bifidobacterium lactis 1 (“BPL1”).

13. The nutritional supplement of claim 1, for inhibiting appetite in the human subject.

14. The nutritional supplement of claim 1, for inhibiting inflammation in the human subject.

15. The nutritional supplement of claim 1, for promoting at least 4 kilograms of weight loss in a human subject upon oral administration at a rate of once or twice a day over a time period of 4 weeks.
